(19) United States
(12) Patent Application Publication
(10) Pub. No.: US 2022/0313167 A1
(43) Pub. Date: Oct. 6, 2022

(54) RECLINER HAVING HEALTHCARE FUNCTION

(71) Applicant: Emma Healthcare Co., Ltd, Seongnam-si (KR)

(72) Inventor: Ryanghee SOHN, Seongnam-si (KR)

(73) Assignee: Emma Healthcare Co., Ltd, Seongnam-si (KR)

(21) Appl. No.: 17/706,267

(22) Filed: Mar. 28, 2022

(30) Foreign Application Priority Data
Mar. 31, 2021 (KR) 10-2021-0042209

(51) Int. Cl. A61B 5/00; A61B 5/11; A61B 5/021

(52) U.S. Cl. CPC A61B 5/6891; A61B 5/1102; A61B 5/7267; A61B 5/7278; A61B 5/7225; A61B 5/742; A61B 5/0002; A61B 5/021; A61B 2562/0247; A61B 2562/164

(57) ABSTRACT

Disclosed is a recliner having a healthcare function, the recliner including a main body unit including a seat portion and a backrest portion, a first BCG sensor unit provided in the seat portion, the first BCG sensor unit being configured to sense a first ballistocardiogram signal, a second BCG sensor unit provided in the backrest portion, the second BCG sensor unit being configured to sense a second ballistocardiogram signal, and a processor configured to pre-process the first ballistocardiogram signal and the second ballistocardiogram signal, to calculate a time difference value in J-peak signals between the pre-processed first ballistocardiogram signal and the pre-processed second ballistocardiogram signal a plurality of times, and to apply the calculated time difference values to a predetermined trained machine learning model as input values in order to calculate a systolic blood pressure value and a diastolic blood pressure value of the user.

This application claims the benefit of Korean Patent Application No. 10-2021-0042209, filed on Mar. 31, 2021, which is hereby incorporated by reference as if fully set forth herein.

### BACKGROUND OF THE INVENTION

### Field of the Invention

The present invention relates to a recliner having a healthcare function, and more particularly to a recliner having a healthcare function capable of measuring blood pressure of a user.

### Discussion of the Related Art

A biosignal includes various kinds of information from which a health state can be checked. Consequently, a biosignal may be measured, and a health state may be predicted based on the measured biosignal. A biosignal mainly used to check a health state in real life is blood pressure. There is a cuff-based method, in which a cuff is wrapped around the forearm to measure blood pressure, as a method of measuring blood pressure. Also, in recent years, research on a cuffless method, in which blood pressure is measured without using a cuff, has been conducted. A sphygmomanometer using the cuffless method indirectly measures blood pressure using biometric information highly related to blood pressure.

A cuff-based blood pressure measurement system has a problem in that a user must carry or wear an instrument, whereby users' utilization thereof is remarkably reduced. On the other hand, the cuffless method, in which blood pressure is measured without using the cuff, has a demerit in that the difference between a blood pressure value indirectly measured using biometric information of a user and an actually measured blood pressure value of the user is large.

A method capable of easily measuring blood pressure without a user wearing a separate instrument in order to improve portability and improving accuracy in blood pressure measurement has not yet been proposed.

The present invention has been made in view of the above problems and proposes a smart healthcare instrument capable of measuring blood pressure without a user wearing or carrying a separate instrument in everyday life while improving accuracy in blood pressure measurement.

### SUMMARY OF THE INVENTION

It is an object of the present invention to provide a recliner having a healthcare function.

It is another object of the present invention to provide a method of measuring blood pressure of a user using a recliner having a healthcare function.

Objects of the present invention are not limited to the aforementioned objects, and other unmentioned objects will be clearly understood by those skilled in the art based on the following description.

In accordance with an aspect of the present invention, the recliner having a healthcare function comprises a main body unit comprising a seat portion and a backrest portion; a first BCG sensor unit provided in the seat portion, the first BCG sensor unit being configured to sense a first ballistocardiogram signal when a user sits in the recliner; a second BCG sensor unit provided in the backrest portion, the second BCG sensor unit being configured to sense a second ballistocardiogram signal when the user leans back on the backrest portion in a state of sitting in the recliner; and a processor configured to: pre-process the first ballistocardiogram signal and the second ballistocardiogram signal sensed by the first BCG sensor unit and the second BCG sensor unit, respectively; calculate a time difference value in J-peak signals between the pre-processed first ballistocardiogram signal and the pre-processed second ballistocardiogram signal a plurality of times; and apply the plurality of calculated time difference values to a predetermined trained machine learning model as input values in order to calculate a systolic blood pressure value and a diastolic blood pressure value of the user.

The first BCG sensor unit or the second BCG sensor unit comprises a thin film type pressure sensor; and a plurality of silicone pads configured to cover opposite surfaces of the thin film type pressure sensor. The thin film type pressure sensor comprises a thin film sensor made of polyvinylidene fluoride (PDVF). The thin film type pressure sensor is provided in the plurality of silicone pads so as to have a Z-shape when the recliner is viewed from above a front.

A surface of each of the seat portion and the backrest portion is covered with leather, the first BCG sensor unit is provided so as to contact an inner surface of the leather that covers the seat portion, and the second BCG sensor unit is provided so as to contact an inner surface of the leather that covers the backrest portion. The pre-processing performed by the processor comprises analog-filtering each of the sensed first ballistocardiogram signal and the sensed second ballistocardiogram signal; amplifying each of the filtered first ballistocardiogram signal and the filtered second ballistocardiogram signal; converting each of the amplified first ballistocardiogram signal and the amplified second ballistocardiogram signal into a digital signal; and digital-filtering each of the first ballistocardiogram signal and the second ballistocardiogram signal converted into the digital signals. The recliner further comprises a display unit configured to display the calculated systolic blood pressure value and the calculated diastolic blood pressure value of the user so as to be recognized by the user. The recliner further comprises a wireless communication unit configured to wirelessly transmit the calculated systolic blood pressure value and the calculated diastolic blood pressure value of the user to a server or a terminal of the user.

In accordance with another aspect of the present invention, the method for measuring blood pressure of a user using a recliner having a healthcare function comprises sensing, by a first BCG sensor unit provided in a seat portion, a first ballistocardiogram signal when the user sits in the recliner; sensing, by a second BCG sensor unit provided in a backrest portion, a second ballistocardiogram signal when the user leans back on the backrest portion in a state of sitting in the recliner; pre-processing, by a processor, the first ballistocardiogram signal and the second ballistocardiogram signal sensed by the first BCG sensor unit and the second BCG sensor unit, respectively; calculating, by the processor, a time difference value in J-peak signals between the pre-processed first ballistocardiogram signal and the pre-processed second ballistocardiogram signal a plurality of times; and applying, by the processor, the plurality of calculated time difference values to a predetermined trained machine learning model as input values in order to calculate a systolic blood pressure value and a diastolic blood pressure value of the user.

The pre-processing comprises analog-filtering each of the sensed first ballistocardiogram signal and the sensed second ballistocardiogram signal; amplifying each of the filtered first ballistocardiogram signal and the filtered second ballistocardiogram signal; converting each of the amplified first ballistocardiogram signal and the amplified second ballistocardiogram signal into a digital signal; and digital-filtering each of the first ballistocardiogram signal and the second ballistocardiogram signal converted into the digital signals.

The method further comprises displaying the calculated systolic blood pressure value and the calculated diastolic blood pressure value of the user so as to be recognized by the user. The method further comprises wirelessly transmitting the calculated systolic blood pressure value and the calculated diastolic blood pressure value of the user to a server or a terminal of the user.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a view illustrating a layer structure of an artificial neural network;

FIG. 2 is a view showing an example of a deep neural network;

FIG. 3 is a view illustratively showing pulse transit time (PTT), which is a feature calculated using ECG, PPG, and BCG;

FIG. 4 is a view illustrating a BCG waveform;

FIG. 5 is a block diagram view provided to explain components of a recliner having a healthcare function according to an embodiment of the present invention capable of measuring blood pressure of a user;

FIG. 6 is a view showing an example of the recliner according to the present invention capable of measuring blood pressure of the user;

FIG. 7 is a view showing an example in which a first BCG sensor unit and a second BCG sensor unit are disposed in a main body unit;

FIG. 8 is a view provided to explain a specific construction of the first BCG sensor unit and the second BCG sensor unit;

FIG. 9 is a view illustrating waveforms of a ballistocardiogram (BCG) signal sensed and measured using the recliner according to the embodiment of the present invention; and

FIGS. 10 and 11 are views provided respectively to explain a machine learning execution method and process for calculating blood pressure of a user in accordance with the present invention.

### DETAILED DESCRIPTION OF THE INVENTION

Hereinafter, preferred embodiments of the present invention will be described in detail with reference to the accompanying drawings. The detailed description disclosed hereinafter together with the accompanying drawings shows exemplary embodiments of the present invention and does not reveal a unique embodiment by which the present invention can be implemented. The following detailed description includes specific details in order to provide complete understanding of the present invention. However, those skilled in the art will appreciate that the present invention can be implemented without such specific details.

In some cases, in order to avoid the concept of the present invention being ambiguous, a well-known structure and apparatus may be omitted, or each structure and apparatus will be shown in the form of a block diagram including core functions thereof. In addition, the same elements are denoted by the same reference numerals throughout this specification.

The present invention proposes an apparatus and method for measuring blood pressure of a user from a biosignal obtained from the user using a machine learning algorithm.

Before describing the present invention, an artificial intelligence (AI), machine learning, and deep learning will be described. As a method of most easily understanding the relationship among the three concepts, three concentric circles may be imagined. Artificial intelligence may be the outermost circle, machine learning may be the middle circle, and deep learning, which leads a current artificial intelligence boom, may be the innermost circle.

The concept of artificial intelligence first appeared in the Dartmouth workshop held by Professor John McCarthy at Dartmouth College, USA in the year of 1956, and has explosively grown in recent years. In particular, artificial intelligence has been further accelerated as the result of introduction of a GPU, which has provided rapid and strong parallel processing performance since 2015. The advent of the big data era with ever-expanding storage capacity and numerous data in all areas, such as images, text, and mapping data, had a great influence on such growth of artificial intelligence.

### Artificial Intelligence—Human Intelligence Implemented by Machine

In 1956, artificial intelligence pioneers dreamt of manufacturing a complex computer having similar characteristics to human intelligence. Artificial intelligence that thinks like a human being while having sense and thinking power of the human being is called a “general Al”, whereas artificial intelligence that can be made at the level of the current technological advancement is included in the concept of “narrow Al”. Narrow AI is characterized in that it is possible to perform specific tasks, such as an image sorting service or a facial recognition function on social media, with greater than human ability.

### Machine Learning—Specific Approach That Implements Artificial Intelligence

Machine learning serves to automatically filter spam in a mailbox. Meanwhile, basically, machine learning analyzes data using an algorithm, learns through analysis, and performs determination or prediction based on what has been learned. Ultimately, therefore, machine learning aims to “train” a computer itself using a large amount of data and the algorithm so as to learn a task execution method, instead of specific guidelines for decision making being directly coded in software. Machine learning came from the concept that early artificial intelligence researchers directly advocated, and decision tree learning, inductive logic programming, clustering, reinforcement learning, and a Bayesian network are included in algorithm schemes. However, none thereof has achieved general AI, which is the final target, and there were many cases in which it was difficult to complete even narrow AI using an initial machine learning approach.

Although machine learning is making great achievements in the field of computer vision at the present time, machine learning encountered the limitation in that a predetermined amount of coding work is accompanied over a process of implementing artificial intelligence, even if there are no specific guidelines. When an image of a stop sign is recognized using a machine learning system, for example, a developer must directly manufacture a border sensing filter that identifies a start part and an end part of an object using a program, shape sensing that determines the surface of the object, and a classifier that recognizes letters such as “S-T-O-P” by coding. Like this, machine learning is operated in a manner in which the image is recognized from the “coded” classifier and the stop sign is “learned” through an algorithm.

An image recognition rate of machine learning is sufficient in performance to be commercialized. In a specific situation in which the sign is invisible due to fog or trees, however, the image recognition rate of machine learning may be reduced. The reason that computer vision and image recognition have not reached to the level of the human being until recently is because of such a recognition rate problem and frequent errors.

### Deep Learning—Technology That Implements Complete Machine Learning

What gave inspiration to an artificial neural network, which is another algorithm that early machine learning researchers made, is the biological characteristics of a human brain, particularly a neuron connection structure. However, the artificial neural network has uniform layer connection and data propagation direction, unlike the brain in which physically adjacent neurons can be connected to each other.

For example, when an image is cut into a great number of tiles and the tiles are input to a first layer of the neural network, the neurons repeat a process of transmitting data to the next layer until a final output is generated by the last layer. A weight indicating input accuracy based on a task that is performed is assigned to each neuron, and after that, all weights are summed, whereby final output is determined. For the stop sign, characteristics of the image, such as the octagonal shape, red color, displayed letters, size, and motion thereof, are finely cut and “inspected”, by the neurons, and the duty of the neural network is to determine whether this is a stop sign. Here, a “provability vector” that predicts the result according to the weights based on sufficient data is utilized.

Deep learning, which is artificial intelligence developed from an artificial neural network, learns data utilizing information input and output layers similar to neurons of a brain. Since even a basic neural network required an awesome amount of operation, however, commercialization of deep learning faced difficulties from the beginning. Nevertheless, research has continued, and parallelization of an algorithm improving the concept of deep learning based on a super computer was successful. The advent of a GPU optimized for parallel operation has epochally accelerated the operating speed of the neural network, whereby artificial intelligence based on true deep learning appeared.

There is a high possibility of the neural network giving a great number of wrong answers during “learning”. Back to the example of the stop sign, hundreds, thousands, or millions of images may be learned in order to accurately adjust neuron input weights so as to always give correct answers irrespective of weather conditions and change of day and night. It can be seen that the neural network has sufficiently learned the stop sign only when this level of accuracy was reached. In the year of 2012, Google and Professor Andrew NG at Stanford University implemented a “deep neural network” constituted by about one billion or more of neurons using 16,000 computers. 10 million images were picked and analyzed from YouTube therethrough, and the computers succeeded in classifying images of people and images of cats. The computers learned a process of recognizing and determining the shape and appearance of the cats displayed on the screens by themselves.

Image recognition ability of a system trained through deep learning has already gone ahead of a human being. In addition, ability of recognizing cancer cells in blood and ability of recognizing tumors through MRI scanning are included in the deep learning area. AlphaGo of Google learned the fundamentals of baduk, which is a Korean strategy board game, and further strengthened the neural network while repeatedly playing games with AI like itself. As the result of the advent of deep learning, practicality of machine learning has been reinforced, and the artificial intelligence area was extended. Deep learning subdivides a task in all supportable manners through a computer system. Deep learning-based technologies, such as a car without a driver, better preventive healthcare, and more accurate movie recommendation, have already been used in our daily life or are about to be put into practice. Deep learning is evaluated as the present and future of artificial intelligence having potential power capable of realizing general AI that appeared in science fiction.

Hereinafter, deep learning will be described in more detail.

Deep learning, which is a kind of artificial neural network (ANN) using a human neural network theory, is a machine learning model or an algorithm set referring to a deep neural network (DNN) configured to have a layered structure in which at least one hidden layer (hereinafter referred to as an “intermediate layer” is provided between an input layer and an output layer. Briefly, deep learning may be an artificial neural network having deep layers.

A human brain is estimated to be constituted by 25 billion nerve cells, each nerve cell (neuron) refers to one neuron constituting the neural network. One neuron includes one cell body, one axon or nurite, which is a protrusion of the cell body, and several dendrites or protoplasmic processes. Information exchange between neurons is performed through a synapse, which is a junction between neurons. Although one neuron is very simple, a group of neurons may have human intelligence. The dendrites are inputs configured to receive signals sent by other neurons, and the axon, which is a portion extending from the cell body, is an output configured to transmit a signal to another neuron. The synapse is a connection portion configured to connect the axon and the dendrites, which transmit signals between neurons, to each other. Signals of the neurons are not unconditionally transmitted but are transmitted only when the intensity of each signal is a predetermined value (threshold) or more. That is, synapses have different connection intensities, and each synapse determines whether to transmit a signal.

An artificial neural network (ANN), which is one field of artificial intelligence, is a mathematical model modeled by imitating the brain structure (neural network) of biology (generally a human being). That is, the artificial neural network is implemented by imitating an information processing and transmission process of a biological neuron. The artificial neural network is implemented similarly to a manner in which a human brain solves problems, and the neural network has excellent parallelism, since the neurons independently operate. In addition, since information is dispersed in many connection lines, no great influence is exerted on all neurons even though some of the neurons have problems, and therefore the artificial neural network is resistant to a predetermined level of errors and has learning ability in a given environment.

A deep neural network, which is a descendant of the artificial neural network, is the latest version of the artificial neural network that goes beyond the existing limits and has achieved successes in areas in which a large number of artificial intelligence technologies suffered failures in the past. When describing modeling an artificial neural network by imitating a biological neural network, biological neurons are modeled as nodes in terms of processing unit, and synapses are modeled as weights in terms of connections, as shown in Table 1 below.

TABLE 1

| Biological neural network | Artificial neural network |
|---|---|
| Cell body | Node |
| Dendrite | Input |
| Axon | Output |
| Synapse | Weight |

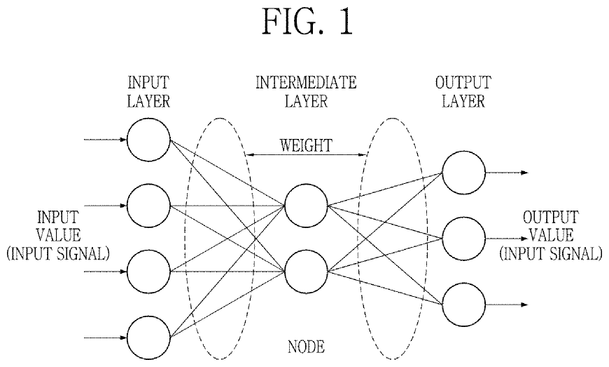

FIG. 1 is a view illustrating a layer structure of an artificial neural network.

Like a plurality of biological neurons of a human being, not a single biological neuron, is connected to each other in order to perform a meaningful task, for an artificial neural network, individual neurons are also connected to each other via synapses, whereby a plurality of layers is connected to each other, wherein connection intensity between the respective layers may be updated using weights. The multilayer structure and connection intensity are utilized in a field for learning and recognition.

The respective nodes are connected to each other via links having weights, and the entire model performs learning while repeatedly adjusting weights. The weights, which are basic means for long-term memory, express importance of the respective nodes. Briefly, the artificial neural network initializes the weights and updates and adjusts weights using a data set to be trained to train the entire model. When a new input value is input after training is completed, an appropriate output value is inferred. The learning principle of the artificial neural network is a process in which intelligence is formed through generalization of experiences, and learning is performed is in a bottom-up manner. When two or more (i.e. 5 to 10) intermediate layers are provided, as shown in FIG. 1, this means that the layers are deepened and is called a deep neural network, and a learning and inference model achieved through the deep neural network may be referred to as deep learning.

The artificial neural network may play a role to some extent even when the artificial neural network has one intermediate layer (generally referred to as a “hidden layer”) excluding input and output. When problem complexity increases, however, the number of nodes or the number of layers must be increased. It is effective to increase the number of layers so as to provide a multilayer model; however, an availably range is restrictive due to limitations in that efficient learning is impossible and the amount of calculation necessary to train the network is large.

As a result of overcoming existing limitations described above, however, the artificial neural network was configured to have a deep structure. Consequently, a complex and expressive model has been constructed, and epochal results have been announced in various fields, such as voice recognition, facial recognition, object recognition, and text recognition.

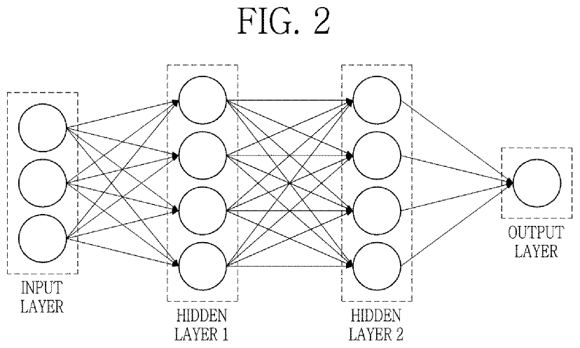

FIG. 2 is a view showing an example of a deep neural network.

A deep neural network (DNN) is an artificial neural network (ANN) having several hidden layers between an input layer and an output layer. The deep neural network is a machine learning model or an algorithm set referring to a deep neural network (DNN) having at least one hidden layer between an input layer and an output layer. Connection of the neural network is achieved from the input layer to the hidden layer and from the hidden layer to the output layer.

The deep neural network may model complex non-linear relationships, like a general artificial neural network. For example, in a deep neural network structure for an object identification model, each object may be expressed as a layer construction of basic elements of an image. At this time, additional layers may rally characteristics of lower layers that are gradually gathered. This characteristic of the deep neural network enables complex data to be modeled using fewer units (nodes) than an artificial neural network that is operated similarly thereto.

Previous deep neural networks were generally designed as feedforward neural networks. In recent research, however, deep learning structures have been successfully applied to a recurrent neural network (RNN). As an example, there are cases in which the deep neural network structure was applied to the field of language modeling. A convolutional neural network (CNN) has been well applied to the field of computer vision, and successful application cases have been well documented. Furthermore, in recent years, the convolutional neural network has been applied to the field of acoustic modeling for automatic speech recognition (ASR), and it is evaluated that the convolutional neural network has been more successfully applied than existing models. The deep neural network may be trained using a standard error back-propagation algorithm. At this time, weights may be updated through stochastic gradient descent using the following equation.

An electrocardiogram (ECG) signal, a photoplethysmogram (PPG) signal, and a ballistocardiogram (BCG) signal may be considered as biometric information highly related to blood pressure. Hereinafter, an ECG sensor unit, a PPG sensor unit, and a BCG sensor unit will be described in brief.

TABLE 2

| Signal | Definition |
|---|---|
| ECG | Recording electrical activity of heart |
| PPG | Recording change in volume of blood |
| | vessel using optical properties |
| BCG | Recording physical vibration generated |
| | when heart contracts |

Electrocardiogram is a curved record of a potential difference generated together with a heart rate as the heart contracts. The heart is distinguished from muscle of other parts in a living body in that the heart automatically and rhythmically contracts. Contraction of heart muscle is like a generator configured to supply electricity to living things. That is, driving force that causes contraction is microcurrent generated in a sinoatrial node of an atrium of the heart. This microcurrent flows through the heart muscle, whereby current flows in the body, and this current is recorded on the surface of the body. A device configured to record the current is called an electrocardiography sensor (ECG sensor), and this record is referred to as electrocardiogram (ECG).

### Photoplethysmogram (PPG)

Next, a photoplethysmography (PPG) sensor will be described as an example of a heart rate sensor. PPG sensor is short for photoplethysmography sensor. Photoplethysmography (PPG) is a pulse wave measurement method that measures the flow rate of blood in a blood vessel using optical characteristics of living tissue in order to check an activity state of the heart or the heart rate. The pulse wave, which is a pulsating waveform generated in the heart while undulating, is measurable through a change in blood flow rate generated according to relaxation and contraction of the heart, i.e. a change in volume of a blood vessel. In photoplethysmography, which is a method of measuring a pulse wave using light, a change in optical properties, such as reflection, absorption, and a transmission rate, of living tissue generated at the time of volume change is sensed and measured by an optical sensor, whereby it is possible to measure pulsation. This method is capable of performing non-invasive pulse measurement, is widely used because of merits, such as miniaturization and convenience in use, and may be used as a biosignal sensor in a wearable device.

### Ballistocardiogram (BCG)

The moment blood discharged from a ventricle of the heart during a cardiac cycle passes through the aorta, the blood transmits reaction to our bodies. A signal measuring vibration (ballistic trajectory) due to a change in blood flow in the heart and the blood vessel related thereto is called ballistocardiogram (BCG). Ballistocardiogram means a signal measuring a ballistic trajectory due to a change in blood flow in the heart and the blood vessel according to contraction and relaxation of the heart, and is an index indicating the activity state of the heart, similarly to electrocardiogram.

Ballistocardiogram is an index indicating the activity state of the heart, similarly to electrocardiogram, and it is known that ballistocardiogram includes information about reflux and abnormal blood flow due to damage to a myocardial function. Consequently, this biosignal has the potential to be clinically utilized, such as evaluation in function of the heart, diagnosis of heart disease (cardiomyopathy), checking of treatment effects, and observation of the degree of recovery. A ballistocardiogram signal may be measured using an acceleration sensor, a load cell sensor, a PVDF film sensor, or an EMFi sensor. Since it is not necessary to attach an electrode to the body when these sensors are used, it is possible to measure a signal in an unconstrained/unconscious state, and the sensors may be usefully utilized in health monitoring for a long time or during everyday life.

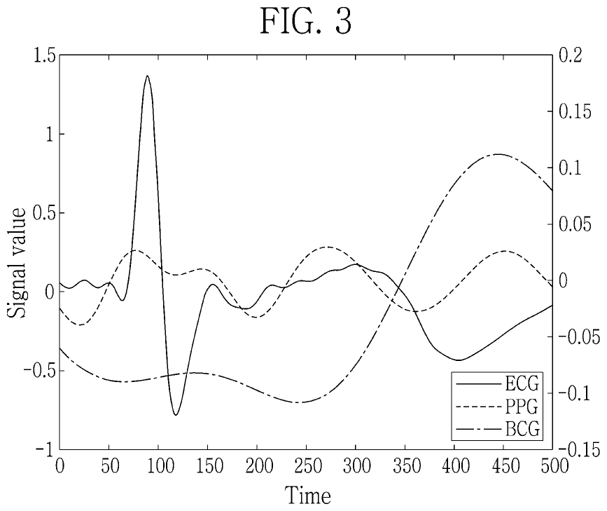

FIG. 3 is a view illustratively showing pulse transit time (PTT), which is a feature calculated using ECG, PPG, and BCG.

There is a method of estimating blood pressure based on a statistical method using pulse transit time (PTT), which is a feature calculated using ECG, PPG, and BCG. However, PTT has demerits in that PTT is affected by the characteristics of the artery and that periodic calibration is necessary due to a change in elasticity of a blood vessel over time. In addition, at the time of measuring a biosignal, noise may be generated depending on the behavior of a user, whereby it is difficult to acquire a feature, and therefore the degree of estimation by a blood pressure estimation algorithm may be reduced. Consequently, a deep learning-based method in which learning is performed through a deep neural network, not a conventional method of acquiring and estimating a feature, may be a good method for solving this.

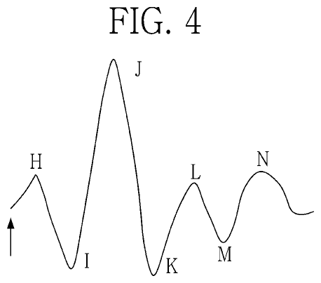

FIG. 4 is a view illustrating a BCG waveform.

Referring to FIG. 4, when the atrium of the heart contracts, a BCG pulse wave is generated, and then the ventricle of the heart contracts. The maximum value and the minimum value of the pulse wave are indicated using letters from H to N. H to K waves corresponding to systole are generated, and L to N waves are generated during diastole corresponding to relaxation of the heart. The waveform of the BCG pulse wave is a combination of forces generated by the heart and blood flow. As a result, it is not easy to relate the waveform to a specific physiological phenomenon. Individually, physical properties of the human body indicate different BCG pulses. The I wave is generated immediately after systole. The I wave is mainly generated by a reactive effect due to acceleration of blood to the aorta. Blood flow in a direction toward the head causes reaction in a direction to the legs. When an aortic arch faces in a downward direction, the direction of blood flow is changed. At this time, reaction in the direction toward the head is generated, and the ballistocardiogram shows a strong J wave. It is estimated that the K wave is generated due to deceleration of the blood flow in systemic circulation. Analysis of diastolic L, M, and N waves is uncertain. It is thought that these waveforms are mainly generated due to a change in direction of the blood flow in peripheral circulation. The diastolic blood flow circulated to the heart so as to fill the atrium of the heart has a small influence on diastolic waveforms. Due to a breathing effect, the amplitudes of I and J waves are generally increased during inspiration, and the amplitudes of I and J waves are decreased during exhalation. The sum of all waveforms indicates a relative cardiac output. Ballistocardiogram is generally measured in a manner in which force directed to the head appears as a rising wave and force directed to the legs appears as a reducing wave.

Hereinafter, a method of calculating a systolic blood pressure value and a diastolic blood pressure value of a user from ballistocardiogram (signal), which is a biosignal of the user directly related to the present invention, using a trained machine learning model (or machine learning algorithm) will be described.

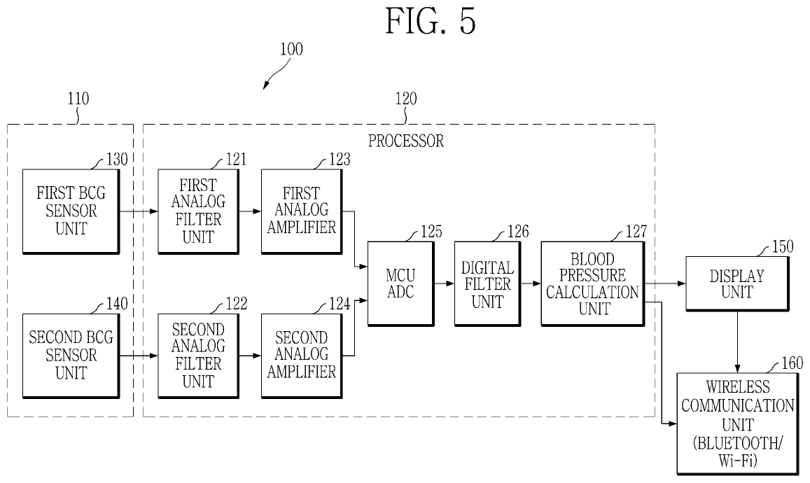

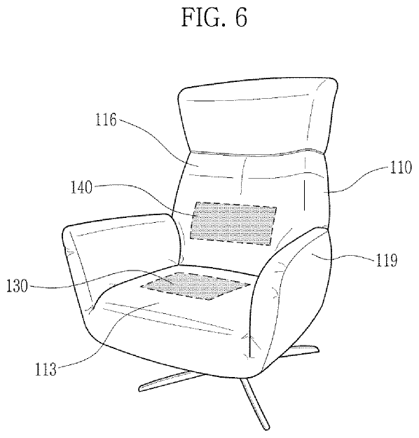

FIG. 5 is a block diagram view provided to explain components of a recliner having a healthcare function according to an embodiment of the present invention capable of measuring blood pressure of a user, and FIG. 6 is a view showing an example of the recliner according to the present invention capable of measuring blood pressure of the user.

Referring to FIG. 5, the recliner 100 capable of measuring blood pressure of the user may include a main body unit 110, a processor 120, a first BCG sensor unit 130 and a second BCG sensor unit 140 provided in the main body unit 110, a display unit 150, and a wireless communication unit 160.

Referring to FIG. 6, the main body unit 110 may include a seat portion 113 configured to support the user when the user takes a seat, a backrest portion 116 configured to support the back of the user when the user takes a seat, an armrest portion 119, and a support portion. As illustrated in FIG. 6, the main body unit 110 of the recliner 100 may be covered with a specific material. A material used as the cover of a chair, such as leather, may be used as the specific material.

As an example, the seat portion 113 may be covered with a leather material, and the first BCG sensor unit 130 is provided inside the leather material. The first BCG sensor unit 130 may be in contact with the inside of the leather material. Since the first BCG sensor unit 130 is provided in the seat portion 113, as described above, the first BCG sensor unit may sense ballistocardiogram (signal) from at least one region of the lower part of the body of the user, such as the inside of a thigh or the hips, when the user takes a seat. It may be preferable for the first BCG sensor unit 130 to sense ballistocardiogram (signal) from a biosignal of the hips.

In the same manner, the backrest portion 116, which is configured to support the upper part of the body of the user, such as the back, may be covered with a leather material, as an example, and the second BCG sensor unit 140 is provided inside the leather material. The backrest portion 116 is configured to be inclined at an angle desired by the user, wherein a basic angle of about 125 degrees may be set. The reason for this is that, when the angle of the backrest portion 116 is about 125 degrees, it is possible to best sense a ballistocardiogram signal from the upper part of the body of the user, such as the back.

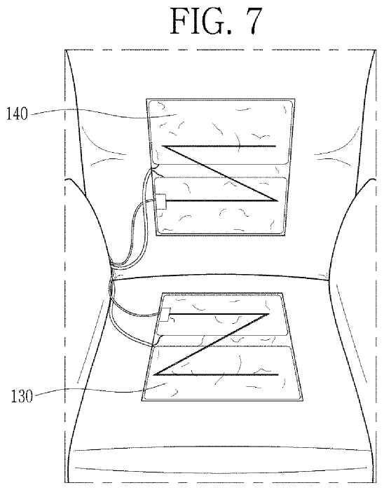

Since the second BCG sensor unit 140 is provided in the backrest portion 116, the second BCG sensor unit may sense a ballistocardiogram signal from the upper part of the body of the user, such as the back, when the user leans back on the backrest portion 116 after taking a seat. An example in which the first BCG sensor unit 130 is disposed in the seat portion 113 and the second BCG sensor unit 140 is disposed in the backrest portion 116 will be described with reference to FIG. 7.

FIG. 7 is a view showing an example in which the first BCG sensor unit 130 and the second BCG sensor unit 140 are disposed in the main body unit 110.

Referring to FIG. 7, the first BCG sensor unit 130 may be installed at a layer immediately under leather, which is the surface of the seat portion 113, in a horizontal direction so as to be located in the vicinity of the middle of the seat portion 113. Here, the horizontal direction may mean a direction in which a PVDF sensor 134 has a Z-shape when the seat portion 113 is viewed from above the front. In a vertical direction, not the horizontal direction, the PVDF sensor 134 may have an N-shape, not a Z-shape, when the seat portion is viewed from above the front. The reason for this is that, when the first BCG sensor unit 130 is disposed in the vertical direction, a signal may not be successfully observed from the hips of the user.

In addition, it is necessary to dispose the first BCG sensor unit 130 so as to contact the inner surface of the leather. The reason of this is that, if an intermediate material, such as sponge, is present between the first BCG sensor unit 130 and the surface of the leather, a signal may not be successfully observed from the hips of the user.

The second BCG sensor unit 140 may be installed at a layer immediately under leather, which is the surface of the backrest portion 116, in a horizontal direction. Here, the horizontal direction may mean a direction in which the PVDF sensor 134 has a Z-shape when the backrest portion 116 is viewed from above the front. In a vertical direction, not the horizontal direction, the PVDF sensor 134 may have an N-shape, not a Z-shape, when the backrest portion is viewed from above the front. The reason for this is that, when the second BCG sensor unit 140 is disposed in the vertical direction, a signal may not be successfully observed from the back of the user.

In addition, it is necessary to dispose the second BCG sensor unit 140 so as to contact the inner surface of the leather. The reason of this is that, if an intermediate material, such as sponge, is present between the second BCG sensor unit 140 and the surface of the leather, a signal may not be successfully observed from the back of the user.

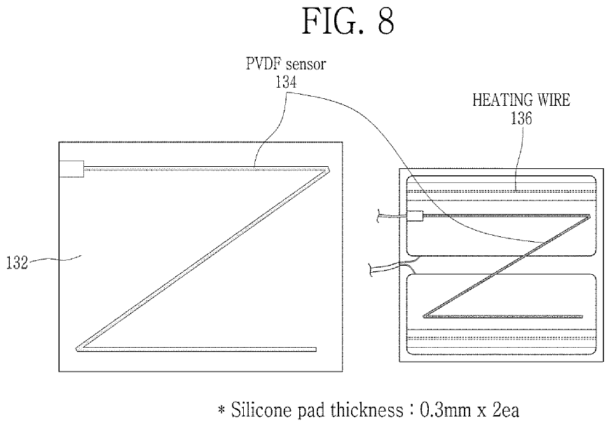

FIG. 8 is a view provided to explain a specific construction of the first BCG sensor unit 130 and the second BCG sensor unit 140.

Referring to FIG. 8, each of the first BCG sensor unit 130 and the second BCG sensor unit 140 may include a thin film type pressure sensor (e.g. a sensor made of a PVDF material or a ceramic thin film material, such as PZT, BST, PN, or PT) 132, silicone pads 134 configured to cover opposite surfaces of the thin film type pressure sensor 132, and a heating wire 136. Hereinafter, the thin film type pressure sensor 132 will be referred to as a PVDF sensor for convenience of description.

Through various experiments, the present invention proposes that opposite surfaces of the PVDF sensor 132 be covered with a silicone material. The reason for this is that, when the silicone pads are used to sense a lower body signal from the hips of the user and an upper body signal from the back of the user, it is possible to acquire the best response characteristics. In particular, each of the silicone pads 134 may have a thickness of 0.3 mm, 0.6 mm, 1.0 mm, or 2.0 mm. It may be preferable for each of the silicone pads to have a thickness of 0.3 mm in order to minimize foreign body feeling when being applied to a recliner product, although there is no great difference in thickness-specific response characteristics.

Each of the first BCG sensor unit 130 and the second BCG sensor unit 140 may include a heating wire 136. In an experimental stage, influence on measurement of a ballistocardiogram signal was not found when heat was applied using the heating wire 236. Consequently, the heating wire 136 may be operated unless heat is applied for a long time.

The first BCG sensor unit 130 may sense a ballistocardiogram signal from a specific region of the user, such as the hips, and the second BCG sensor unit 140 may sense a ballistocardiogram signal from the back of the user. Hereinafter, a ballistocardiogram signal sensed from the hips of the user will be referred to as a first ballistocardiogram signal, and a ballistocardiogram signal sensed from the back of the user will be referred to as a second ballistocardiogram signal.

Referring back to FIG. 5, the processor 120 may include a first analog filter unit 121, a second analog filter unit 122, a first analog amplifier 123, a second analog amplifier 124, an MCU ADC 125, a digital filter unit 126, and a blood pressure calculation unit 127.

The first analog filter unit 121 and the second analog filter unit 122 receive the sensed first ballistocardiogram signal (corresponding to BCG_seat_raw data) and the sensed second ballistocardiogram signal (corresponding to BCG_seat_raw data) from the first BCG sensor unit 130 and the second BCG sensor unit 140, respectively, and perform analog-filtering on the received signals. The first analog amplifier 123 and the second analog amplifier 124 receive the filtered first ballistocardiogram signal and the filtered second ballistocardiogram signal from the first analog filter unit 121 and the second analog filter unit 122, respectively, and amplify the received signals. The MCU ADC 125 converts the first ballistocardiogram signal and the second ballistocardiogram signal amplified by the first analog amplifier 123 and the second analog amplifier 124, respectively, into digital signals. The digital filter unit 126 performs digital filtering on the first ballistocardiogram signal and the second ballistocardiogram signal converted into the digital signals.

A series of processes performed in the processor 120 may be expressed as pre-processing (pre-filtering). As described above, the processor 120 performs pre-processing on the first ballistocardiogram signal and the second ballistocardiogram signal sensed by the first BCG sensor unit 130 and the second BCG sensor unit 140, respectively.

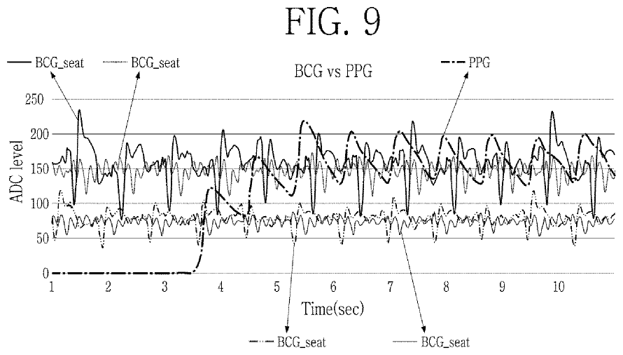

FIG. 9 is a view illustrating waveforms of a ballistocardiogram (BCG) signal sensed and measured using the recliner 100 according to the embodiment of the present invention.

Referring to FIG. 9, a waveform indicated by BCG_seat_raw is a waveform sensed by the first BCG sensor unit 130 and transmitted to the first analog filter unit 121 in FIG. 5, and a waveform indicated by BCG_back_raw is a waveform transmitted to the second analog filter unit 122. In addition, a waveform indicated by BCG_seat and a waveform indicated by BCG_back are information about signals input to the blood pressure calculation unit 127 in FIG. 5.

In FIG. 5, the blood pressure calculation unit 127 is shown as a module included in the processor 120; however, the blood pressure calculation unit may be included in a separate processor. The blood pressure calculation unit 127 may calculate a time difference value between the pre-processed first ballistocardiogram signal and the pre-processed second ballistocardiogram signal. Here, the time difference value may mean a time difference value in J-peak signals between the pre-processed first ballistocardiogram signal and the pre-processed second ballistocardiogram signal, as an example. While a user sits in the recliner 100, the first BCG sensor unit 130 and the second BCG sensor unit 140 sense a first ballistocardiogram signal and a second ballistocardiogram signal, respectively, for a predetermined time (e.g. 15 minutes), and the processor 120 pre-processes the sensed first ballistocardiogram signal and the sensed second ballistocardiogram signal. The blood pressure calculation unit 127 calculates a time difference value in J-peak signals between the first ballistocardiogram signal and the second ballistocardiogram signal measured for the predetermined time and pre-processed. Calculation may be performed a plurality of times for 15 minutes. In the present invention, as an example, the time difference value is calculated 9 times in 15 minutes.

Afterwards, the blood pressure calculation unit 127 may calculate a blood pressure value (a systolic blood pressure value and/or a diastolic blood pressure value) of the user using a machine learning (e.g. deep learning) model. Hereinafter, a machine learning execution method and process will be described.

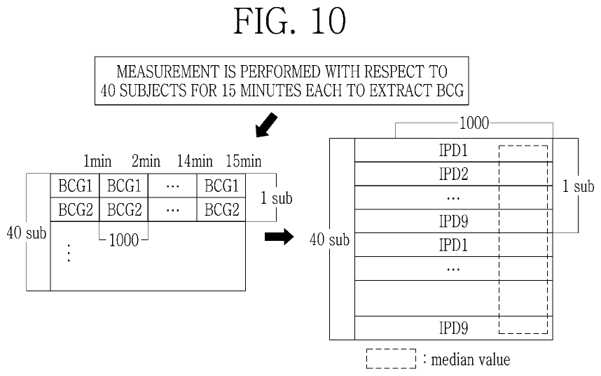

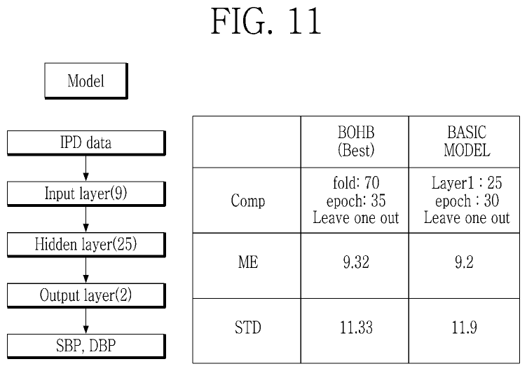

FIGS. 10 and 11 are views provided respectively to explain a machine learning execution method and process for calculating blood pressure of the user in accordance with the present invention.

Referring to FIG. 10, as an example, the first BCG sensor unit 130 extracts or senses a first ballistocardiogram signal (BCG 1) and the second BCG sensor unit 140 extracts or senses a second ballistocardiogram signal (BCG 2) with respect to 40 subjects for 15 minutes each, for example in units of 1 minute. After being pre-processed by the processor 120, the blood pressure calculation unit 127 calculates a time difference value (IPD) in J-peak signals between the pre-processed first ballistocardiogram signal and the pre-processed second ballistocardiogram signal. In FIG. 10, IPD 1 to IPD 9 are shown, since calculation is performed 9 times.

Referring to FIG. 11, the blood pressure calculation unit 127 applies time difference values (IPD) in J-peak signals between a plurality of pre-processed first ballistocardiogram signals and a plurality of pre-processed second ballistocardiogram signals to a trained deep learning model (algorithm) as input values. For example, in FIG. 11, 9 time difference values are input to the trained deep learning model (algorithm). Subsequently, the blood pressure calculation unit 127 may calculate a systolic blood pressure value (SBP) and a diastolic blood pressure value (DBP) of the user as outputs using the deep learning model. At this time, various models, such as a CNN, were studied and a model having high performance was derived as the trained deep learning model. Thereamong, ME was reduced to 9 through model fine-tuning using BOHB. BOHB, which is a combination of a Bayesian optimization technique and hyperband, is a model in which Tree Parzen Estimate is used for Bayesian optimization in order to improve brevity and calculation efficiency. As shown in FIG. 11, fold and epoch values were applied to the trained deep learning model.

The time difference value (IPD) in J-peak signals between the pre-processed first ballistocardiogram signal and the pre-processed second ballistocardiogram signal is calculated a plurality of times and the calculated values are input to train the deep learning model. At the same time, the user may measure blood pressure values (a systolic blood pressure value and a diastolic blood pressure value) using a conventional forearm sphygmomanometer, and may set the measured values as output values. The deep learning model is trained through the above process.

The display unit 150 displays the systolic blood pressure value and the diastolic blood pressure value of the user calculated by the blood pressure calculation unit 127 so as to be recognized by the user. The display unit 150 may be connected to the main body unit 110. However, the present invention is not limited thereto.

In addition, the wireless communication unit 160 may wirelessly transmit the systolic blood pressure value and the diastolic blood pressure value of the user calculated by the blood pressure calculation unit 127 to a server (server PC) or a terminal (smartphone or an application in the smartphone) of the user through Bluetooth or Wi-Fi.

As described above, while the user rests in the recliner at home, blood pressure of the user is calculated and provided, whereby healthcare of the user may be satisfactorily achieved in everyday life.

In addition, the difference between a blood pressure value measured/calculated using the user's blood pressure measurement/calculation method proposed by the present invention and an actual blood pressure value measured by any other wearable device is small, whereby it is possible to improve accuracy in healthcare of the user.

The processor 120 may also be referred to as a controller, a microcontroller, a microprocessor, or a microcomputer. Meanwhile, the processor 120 may be implemented by hardware, firmware, software, or a combination thereof. When an embodiment of the present invention is implemented using hardware, application specific integrated circuits (ASICs), digital signal processors (DSPs), digital signal processing devices (DSPDs), programmable logic devices (PLDs), or field programmable gate arrays (FPGAs), which are configured to perform the present invention, may be provided in the processor 120.

As is apparent from the above description, blood pressure of a user is calculated and provided while the user rests in a recliner at home, whereby healthcare of the user may be satisfactorily achieved in everyday life.

In addition, the difference between a blood pressure value measured/calculated using a user's blood pressure measurement/calculation method proposed by the present invention and an actual blood pressure value measured by any other wearable device is small, whereby it is possible to improve accuracy in healthcare of the user.

It should be noted that the effects of the present invention are not limited to the effects mentioned above, and other unmentioned effects will be clearly understood by those skilled in the art from the above description of the present invention.

The embodiments described above are predetermined combinations of elements and features of the present invention. Each element or feature must be considered to be optional unless explicitly mentioned otherwise. Each element or feature may be implemented in a state of not being combined with another element or feature. In addition, some elements and/or features may be combined to constitute an embodiment of the present invention. The sequence of operations described in the embodiments of the present invention may be changed. Some elements or features in a certain embodiment may be included in another embodiment, or may be replaced with corresponding elements or features in another embodiment. It is obvious that claims having no explicit citation relationship may be combined to constitute an embodiment or may be included as a new claim by amendment after application.

Those skilled in the art will appreciate that the present invention may be embodied in other specific forms than those set forth herein without departing from essential characteristics of the present invention. The above description is therefore to be construed in all aspects as illustrative and not restrictive. The scope of the invention should be determined by reasonable interpretation of the appended claims and all changes coming within the equivalency range of the invention are intended to be within the scope of the invention.

What is claimed is:

1. A recliner having a healthcare function, the recliner comprising:
a main body unit comprising a seat portion and a backrest portion;
a first BCG sensor unit provided in the seat portion, the first BCG sensor unit being configured to sense a first ballistocardiogram signal when a user sits in the recliner;
a second BCG sensor unit provided in the backrest portion, the second BCG sensor unit being configured to sense a second ballistocardiogram signal when the user leans back on the backrest portion in a state of sitting in the recliner; and
a processor configured to:
pre-process the first ballistocardiogram signal and the second ballistocardiogram signal sensed by the first BCG sensor unit and the second BCG sensor unit, respectively;
calculate a time difference value in J-peak signals between the pre-processed first ballistocardiogram signal and the pre-processed second ballistocardiogram signal a plurality of times; and
apply the plurality of calculated time difference values to a predetermined trained machine learning model as input values in order to calculate a systolic blood pressure value and a diastolic blood pressure value of the user.

2. The recliner according to claim 1, wherein the first BCG sensor unit or the second BCG sensor unit comprises:
a thin film type pressure sensor; and
a plurality of silicone pads configured to cover opposite surfaces of the thin film type pressure sensor.

3. The recliner according to claim 2, wherein the thin film type pressure sensor comprises a thin film sensor made of polyvinylidene fluoride (PDVF).

4. The recliner according to claim 2, wherein the thin film type pressure sensor is provided in the plurality of silicone pads so as to have a Z-shape when the recliner is viewed from above a front.

5. The recliner according to claim 1, wherein
a surface of each of the seat portion and the backrest portion is covered with leather,
the first BCG sensor unit is provided so as to contact an inner surface of the leather that covers the seat portion, and
the second BCG sensor unit is provided so as to contact an inner surface of the leather that covers the backrest portion.

6. The recliner according to claim 1, wherein pre-processing performed by the processor comprises:
analog-filtering each of the sensed first ballistocardiogram signal and the sensed second ballistocardiogram signal;
amplifying each of the filtered first ballistocardiogram signal and the filtered second ballistocardiogram signal;
converting each of the amplified first ballistocardiogram signal and the amplified second ballistocardiogram signal into a digital signal; and
digital-filtering each of the first ballistocardiogram signal and the second ballistocardiogram signal converted into the digital signals.

7. The recliner according to claim 1, further comprising:
a display unit configured to display the calculated systolic blood pressure value and the calculated diastolic blood pressure value of the user so as to be recognized by the user.

8. The recliner according to claim 1, further comprising:
a wireless communication unit configured to wirelessly transmit the calculated systolic blood pressure value and the calculated diastolic blood pressure value of the user to a server or a terminal of the user.

9. A method for measuring blood pressure of a user using a recliner having a healthcare function, the method comprising:
sensing, by a first BCG sensor unit provided in a seat portion, a first ballistocardiogram signal when the user sits in the recliner;
sensing, by a second BCG sensor unit provided in a backrest portion, a second ballistocardiogram signal when the user leans back on the backrest portion in a state of sitting in the recliner;
pre-processing, by a processor, the first ballistocardiogram signal and the second ballistocardiogram signal sensed by the first BCG sensor unit and the second BCG sensor unit, respectively;
calculating, by the processor, a time difference value in J-peak signals between the pre-processed first ballistocardiogram signal and the pre-processed second ballistocardiogram signal a plurality of times; and
applying, by the processor, the plurality of calculated time difference values to a predetermined trained machine learning model as input values in order to calculate a systolic blood pressure value and a diastolic blood pressure value of the user.

10. The method according to claim 9, wherein the pre-processing comprises:
analog-filtering each of the sensed first ballistocardiogram signal and the sensed second ballistocardiogram signal;
amplifying each of the filtered first ballistocardiogram signal and the filtered second ballistocardiogram signal;
converting each of the amplified first ballistocardiogram signal and the amplified second ballistocardiogram signal into a digital signal; and
digital-filtering each of the first ballistocardiogram signal and the second ballistocardiogram signal converted into the digital signals.

11. The method according to claim 9, further comprising displaying the calculated systolic blood pressure value and the calculated diastolic blood pressure value of the user so as to be recognized by the user.

12. The method according to claim 9, further comprising wirelessly transmitting the calculated systolic blood pressure value and the calculated diastolic blood pressure value of the user to a server or a terminal of the user.
